(19) United States
(12) Patent Application Publication
(10) Pub. No.: US 2018/0158542 A1
(43) Pub. Date: Jun. 7, 2018

(54) SENSOR CONTROL SYSTEM AND METHOD FOR HEALTH CARE

(71) Applicants: Keun Jei KIM, Seoul (KR); Young Kyu KIM, Seoul (KR); Dong Keon KIM, Seoul (KR)

(72) Inventors: Keun Jei KIM, Seoul (KR); Young Kyu KIM, Seoul (KR); Dong Keon KIM, Seoul (KR)

(21) Appl. No.: 15/567,147

(22) PCT Filed: Mar. 14, 2016
(86) PCT No.: PCT/KR2016/002515

§ 371 Date: Oct. 17, 2017

(30) Foreign Application Priority Data
Apr. 22, 2015 (KR) 10-2015-0056808

(51) Int. Cl. G16H 15/00; A61B 5/00; A61B 5/053

(52) U.S. Cl. CPC G16H 15/00; A61B 5/6807; A61B 2562/04; A61B 5/053; A61B 5/0022; A61B 5/681

(57) ABSTRACT

Disclosed herein are a sensor control system and method for health care. The sensor control system may include: multiple sensor modules, each of the multiple sensor modules being modularized to include at least one sensor, being attached to or detached from a connection interface, and sending information sensed by the at least one sensor to a sensor controller; the connection interface, which the multiple sensor modules are attached to or detached from, and which relays communication between the sensor controller and the multiple sensor modules; and the sensor controller for requesting information from the multiple sensor modules through the connection interface according to a schedule and for processing sensing information received from the multiple sensor modules, wherein the sensor control system may be included in an object worn on a specific part of a human body.

### TECHNICAL FIELD

Embodiments of the present invention relate to a sensor control system and method for health care.

### BACKGROUND ART

In a broad sense, “health care” means the business of general health management, in which the prevention and management of disease is added to existing medical treatment services. In a narrow sense, “health care” refers to activities such as remote medical examinations, health consultation visits, and the like.

Sensors necessary for health care need to measure and provide various pieces of information by themselves rather than by users, as objects in the Internet of Things (IoT) environment do by themselves. To this end, the sensors are worn on the body of a user in the form of a wearable device included in a watch, shoes, or the like, in order to sense information about the user. For example, Korean Patent Application Publication No. 10-2012-0130306 discloses a health care system using smart shoes, which analyzes an emergency state, the number of burned calories and the center of mass based on the exercise pattern of the wearer of the shoes, and thereby may provide emergency calling services for responding to an emergency state, weight management services and posture correction services.

However, the conventional art has a problem related to a spatial limitation that results from attaching various sensors to a wearable product (for example, a watch or the above-mentioned shoes or insoles). Furthermore, with the increase in the number of sensors and the number of times that information is sensed, power consumption for operating the sensors may increase. For example, in the conventional art, because information sensed by the sensors is periodically transmitted to a mobile communication terminal of a user, the power consumption may excessively increase.

### DISCLOSURE

### Technical Problem

There is provided a sensor control system and method in which multiple sensors are grouped, the sensors are modularized based on a group, and a module of the necessary sensor group is provided in the form of being attachable to and detachable from a wearable device, whereby a spatial limitation may be overcome.

There is provided a sensor control system and method that may provide a connection interface by which users may easily add a grouped sensor module to a wearable device that includes a sensor controller, without prior knowledge.

There is provided a sensor control system and method that may reduce power consumption for operating sensors by automatically controlling an information request cycle depending on the kind of sensor module that is attached to a sensor controller.

### Technical Solution

There is provided a sensor control system including: multiple sensor modules, each of the multiple sensor modules being modularized to include at least one sensor, being attached to or detached from a connection interface, and sending information sensed by the at least one sensor to a sensor controller; the connection interface, which the multiple sensor modules are attached to or detached from, and which relays communication between the sensor controller and the multiple sensor modules; and the sensor controller for requesting information from the multiple sensor modules through the connection interface according to a schedule and for processing sensing information received from the multiple sensor modules, wherein the sensor control system is included in an object worn on a specific part of a human body.

According to an aspect, the multiple sensor modules may comprise one or more analog sensor modules individually attached to or detached from the connection interface, and multiple digital sensor modules attached to or detached from the connection interface in series, wherein each of the digital sensor modules may comprise an integrated interface for a sensor included therein, and may communicate with the connection interface through the integrated interface, or may communicate with an integrated interface of another digital sensor module attached thereto in series through the integrated interface.

According to another aspect, the sensor controller may manage information about the at least one sensor included in each of the multiple sensor modules in connection with an identifier of the sensor module, and may send an information request that includes the identifier to the multiple sensor modules through the connection interface.

According to a further aspect, the multiple sensor modules may comprise multiple digital sensor modules attached to the connection interface in series, and in response to an information request sent by the sensor controller, each of the multiple digital sensor modules may compare its identifier with an identifier included in the information request, and may send sensing information to the sensor controller if the two identifiers are identical to each other, or may deliver the information request to a next digital sensor module attached thereto in series if the two identifiers differ from each other.

According to yet another aspect, if the at least one sensor includes an electrode that requires electric contact with a skin of a user, at least one of the object and a supplementary object, worn together with the object, may include conductive material between the electrode and the skin of the user.

According to still another aspect, the connection interface may send the sensor controller a signal related to a sensor module detached from the connection interface or a signal related to a sensor module newly attached to the connection interface, among the multiple sensor modules, and the sensor controller may detect the detached sensor module or the newly attached sensor module using the received signal.

According to still another aspect, the sensor control system may further include a network interface for transmitting the sensing information or information acquired by processing the sensing information to a user terminal, wherein the schedule may be preset using information about a kind of a sensor of the multiple sensor modules attached to the connection interface and information about a user, which is input to the user terminal.

There is provided a sensor control method of a sensor control system, the method including: managing information about sensors included in multiple sensor modules in connection with identifiers of the multiple sensor modules, each of the multiple sensor modules being modularized to include at least one sensor and being attached to or detached from a connection interface included in the sensor control system; sending an information request that includes the identifier to the multiple sensor modules according to a schedule, the multiple sensor modules determining whether to reply to the information request by comparing their identifier with the identifier included in the information request; and collecting sensing information delivered through the multiple sensor modules and the connection interface in response to the information request, wherein the sensor control system is included in an object worn on a specific part of a human body.

### Advantageous Effects

Multiple sensors are grouped, the sensors are modularized based on a group, and a module of a necessary sensor group is provided in the form of being attachable to or detachable from a wearable device, whereby a spatial limitation may be overcome.

A connection interface may be provided in order for users to easily add a grouped sensor module to a wearable device that includes a sensor controller, without prior knowledge.

An information request cycle is automatically controlled depending on the kind of sensor module attached to a sensor controller, whereby power consumption for operating sensors may be reduced.

### DESCRIPTION OF DRAWINGS

FIG. 1 is a view illustrating the internal configuration of a sensor control system in an embodiment of the present invention;

FIG. 2 is a view illustrating the process of collecting sensing information in an embodiment of the present invention;

FIG. 3 is a flowchart illustrating a sensor control method in an embodiment of the present invention;

FIG. 4 is a view illustrating an example of shoes to which a sensor control system is attached in an embodiment of the present invention; and

FIG. 5 is a view illustrating an example of a smart watch to which a sensor control system is attached in an embodiment of the present invention.

### BEST MODE

Hereinafter, an embodiment of the present invention is described in detail with reference to the accompanying drawings.

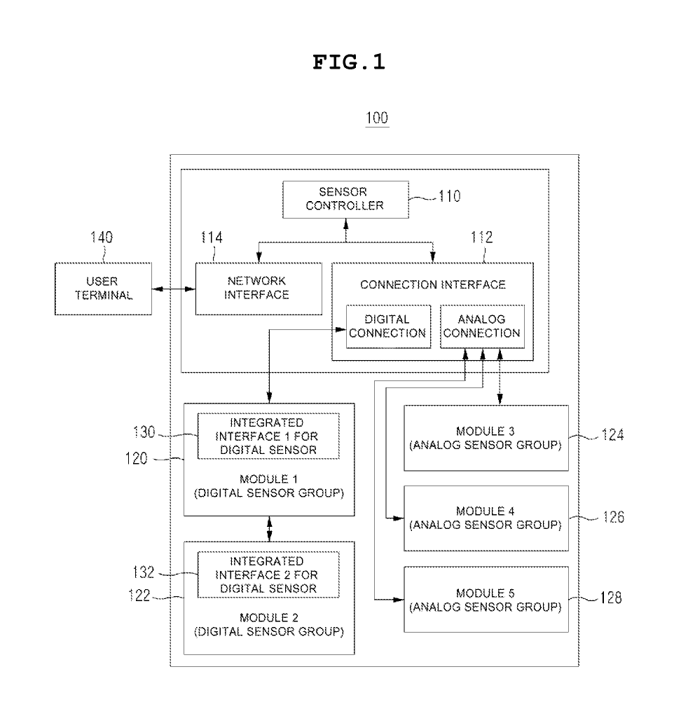

FIG. 1 is a view illustrating the internal configuration of a sensor control system in an embodiment of the present invention. The sensor control system 100 according to the embodiment may be a device for controlling sensors for health care by being included in clothes, shoes, insoles, glasses, a watch or the like.

FIG. 1 shows an example of a sensor control system 100 that includes a sensor controller 110, a connection interface 112, a network interface 114, and multiple modules 120, 122, 124, 126 and 128.

The sensor controller 110 may receive information, sensed by at least one sensor included in each of the multiple modules 120, 122, 124, 126 and 128, from the multiple modules 120, 122, 124, 126 and 128, and may process the received information, wherein each of the modules may be connected to the sensor connection interface 120 in an attachable and detachable manner.

The multiple modules 120, 122, 124, 126 and 128 represent an example in which two modules 120 and 122 for a digital sensor group and three modules 124, 126 and 128 for an analog sensor group are selectively attached. Each of the modules 120 and 122 for the digital sensor group may include at least one digital sensor, and each of the modules 124, 126 and 128 for the analog sensor group may include at least one analog sensor.

Embodiments of the present invention may use at least some currently known sensors for health care, and these sensors may be categorized into analog sensors and digital sensors. Here, the analog sensors may be categorized into multiple analog sensor groups, and each of the analog sensor groups may form a module. The digital sensors may also be categorized into multiple digital sensor groups, and each of the digital sensor groups may form a module. For example, sensors for measuring bioimpedance by being in contact with a user may be modularized as a single group, sensors for measuring and using acceleration may be modularized as a single group, or sensors for collecting information about the external environment (for example, sensors for measuring the concentrations of carbon dioxide, carbon monoxide, nitrogen dioxide, ozone, fine dust, ultra-fine dust, and the like, or sensors for measuring temperature, humidity, and the like) may be modularized as a single group.

The modules 124, 126 and 128 for the analog sensor group may be individually connected to the connection interface 112. Also, the modules 120 and 122 for the digital sensor group may include an integrated interface for digital sensors (an integrated interface 1 130 for a digital sensor and an integrated interface 2 132 for a digital sensor in FIG. 1), and may be attached to the connection interface 112 through these integrated interfaces. Here, the modules 120 and 122 for the digital sensor group may be connected in series to the connection interface 112. For example, when a new module (not illustrated) for the digital sensor group is additionally attached, the new module may be connected in series to the connection interface 112 via the module 2 122 and the module 1 120.

The sensor controller 110 receives information sensed by the multiple sensors included in the multiple modules 120, 122, 124, 126 and 128 through the connection interface 112. Then, the information may be sent to an external user terminal 140 through the network interface 114, after being processed or without being processed. The user terminal 140 may be a user's device, such as a smart phone, for managing information about the user, sensing information and the like.

In another embodiment, the sensor controller 110 may further include an output unit (for example, a display) for processing and outputting the received sensing information. Selectively, the output unit may substitute for the network interface 114.

The connection interface 112 may send a signal related to detachment of a module from the connection interface 112 or a signal related to a module newly attached thereto to the sensor controller 110, the detached module and the attached module being selected from among the multiple modules 120, 122, 124, 126 and 128. Accordingly, the sensor controller may detect the detachment of the module or the newly attached module using the received signal.

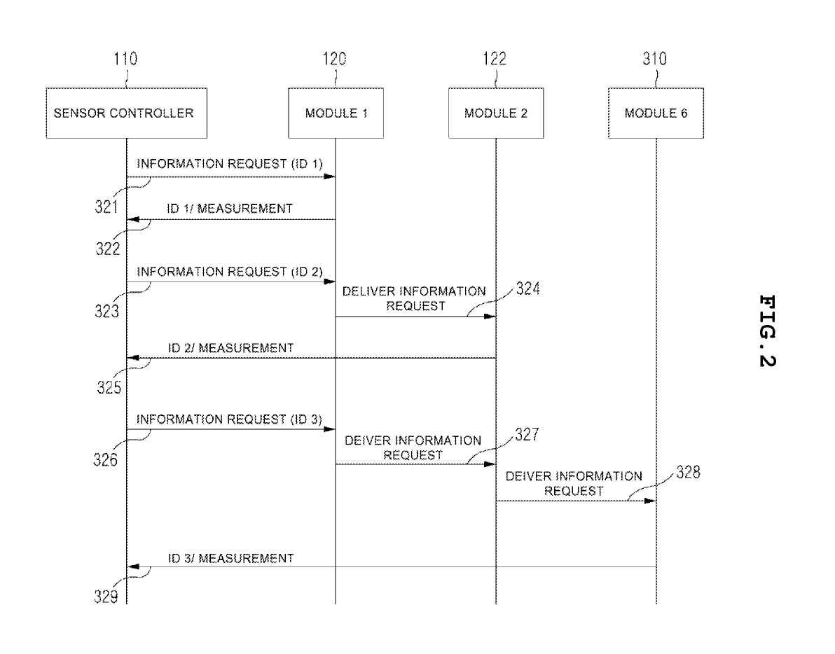

FIG. 2 is a view illustrating the process of collecting sensing information in an embodiment of the present invention. FIG. 2 shows an example of the process of collecting sensing information from the three modules 120, 122 and 310 for the digital sensor group. Here, it is assumed that the three modules 120, 122, and 310 are connected in series to the connection interface 112 in the order of module 1 120, module 2 122 and module 3 310.

The following Table 1 is a table in which the identifiers of the modules 120, 122 and 310 are stored in connection with the kinds of the sensors in the modules.

TABLE 1

| Identifier | Kind of sensor |
|---|---|
| ID 1 | sensors A, B and C |
| ID 2 | sensors D and E |
| ID 3 | Sensors F, G, H and I |

Here, it is assumed that the identifier of the module 1 120 is ID 1, that the identifier of the module 2 122 is ID 2, and that the identifier of the module 3 310 is ID 3. The information about the kind of sensors is automatically detected when the modules 120, 122 and 310 are connected to the connection interface 112, and may be stored in the table, along with the identifiers. For example, when a new module 6 310 is connected in series to the connection interface 112 via the module 2 122, the new module 6 310 may send the identifier thereof and information about the kind of the sensors therein to the sensor controller 110 through the connection interface 112. Accordingly, the sensor controller 110 may match the received identifier and the kind of sensors to each other and store them in the table. Therefore, a user may use various kinds of modules by connecting them in an attachable and detachable manner even if the user has no prior knowledge of sensors or the connection of the sensors.

The sensor controller 110 may detect the sensors included in the inserted module using the information about the kind of the sensors, and may control an information request cycle depending on the inserted module, whereby power consumption for operating the sensors may be reduced.

In the first process 321, in order to request information from the module 1 120, the sensor controller 110 may send an information request that includes an ID 1, which is the identifier of the module 1 120. This request may be delivered to the module 1 120 through the connection interface 112.

In the second process 322, the module 1 120 may compare its identifier ID 1 with the identifier ID 1 included in the information request. Here, because the two identifiers are the same as each other, the module 1 120 may send a measurement, which is sensing information, along with its identifier ID 1, to the sensor controller 110. Here, the identifier and the measurement may be sent to the sensor controller 110 through the connection interface 112, and using the identifier ID 1, which is received along with the measurement, the sensor controller 110 may identify the module that sent the received measurement.

In the third process 323, in order to request information from the module 2 122, the sensor controller 110 may send an information request that includes an ID 2, which is the identifier of the module 2 122.

In the fourth process 324, the module 1 120 may compare its identifier ID 1 with the identifier ID 2 included in the information request. Here, because the identifier ID 2 included in the information request differs from the identifier ID 1 of the module 1 120, the module 1 120 may deliver the information request to the module 2 122, which is the next module in the series connection.

In the fifth process 325, the module 2 122 may compare its identifier ID 2 with the identifier ID 2 included in the information request. Here, because the two identifiers are the same as each other, the module 2 122 may send a measurement, which is sensing information, along with its identifier ID 2, to the sensor controller 110. Here, the identifier and the measurement may be sent to the sensor controller 110 via the module 1 120 and the connection interface 112. Similarly, using the identifier ID 2, which is received along with the measurement, the sensor controller 110 may identify the module that sent the received measurement.

In the sixth process 326, in order to request information from the module 3 310, the sensor controller 110 may send an information request that includes an ID 3, which is the identifier of the module 3 310.

In the seventh process 327, the module 1 120 may compare its identifier ID 1 with the identifier ID 3 included in the information request. Here, because the identifier ID 3 included in the information request differs from the identifier ID 1 of the module 1 120, the module 1 120 may deliver the information request to the module 2 122, which is the next module in the series connection.

In the eighth process 328, the module 2 122 may compare its identifier ID 2 with the identifier ID 3 included in the information request. Here, because the identifier ID 3 included in the information request differs from the identifier ID 2 of the module 2 122, the module 2 122 may deliver the information request to the module 3 310, which is the next module in the series connection.

In the ninth process 329, the module 3 310 may compare its identifier ID 3 with the identifier ID 3 included in the information request. Here, because the two identifiers are the same as each other, the module 3 310 may send a measurement, which is sensing information, along with its identifier ID 3, to the sensor controller 110. Here, the identifier and the measurement may be sent to the sensor controller 110 via the module 1 120, the module 2 122 and the connection interface 112. Similarly, using the identifier ID 3, which is received along with the measurement, the sensor controller 110 may identify the module that sent the received measurement.

As described above, the sensor controller 110 sends an information request to individual modules. Then, using the received identifier and measurement, the sensor controller 110 may identify not only the module in which the measurement was acquired but also the sensors that measured the received measurement using the above-described Table 1.

The sensor controller 110 may automatically set configuration details for measurement (the sensing method, the sensing time, the number of times that information is to be sensed, and the like) depending on the kind of module that is attached (information about the kind of the sensors) or information input from a user (for example, information about the physical condition of the user). Also, a user may change the configuration details of the sensor controller 110 using the user terminal 140.

The sensor controller 110 may control the information request cycle based on these configuration details. As described above, because information is requested for each module according to the controlled cycle, the consumption of power for operating the sensors may be optimized.

The power source of the sensor control system 100 may be provided in the form of a battery, and may be included in the sensor control system 100 along with the sensor controller 110, or may be provided in the form of a detachable module. The battery may be implemented to be rechargeable. For example, if the sensor control system 100 is implemented to be included in shoes (or insoles), the battery may be implemented to be automatically charged by arranging the shoes (or insoles) on a wireless charger, or the battery may be detached, charged using a cable, and then be attached again.

Hereinafter, an embodiment in which the sensor control system 100 manages biometric sensors by being included in shoes or insoles is described in more detail.

For example, a biometric sensor may include various sensors, such as: (1) a pressure sensor for measuring weight, (2) an acceleration sensor for work rate measurement and gait correction, (3) a bioimpedance sensor for measuring body fat and body water, (4) a heart rate sensor and a Photo-plethysmography (PPG) array sensor for measuring a pulse, (5) a vibration sensor for measuring impact force on a body, (6) a temperature sensor and pressure sensor for measuring body temperature and the pressure applied to soles, (7) a Galvanic Skin Reflex (GSR) sensor for measuring psychological arousal/stress, (8) an insole sensor for measuring a change in the power applied to the feet, (9) a piezo sensor for measuring a heart rate, (10) a glucose sensor for measuring blood glucose, and the like. These sensors may be modularized into multiple modules, and a user may use the modules by attaching them to the sensor control system 100 depending on the purpose.

Because some sensors require electric contact with user's skin, the sensor may be provided such that at least some of the parts that contact the user's skin in the sensor are conductive. For example, in order for an electrode included in a bioimpedance sensor arranged in the insole to be in electric contact with the user's skin, conductive socks (functional socks) may be provided. Conductive socks are made such that some parts thereof have conductivity, and may provide electric contact between the electrode and the user's skin. Other objects worn on a specific part of the body of a user, such as other clothes or the like, may have conductivity.

Sensing information received by the sensor control system 100 may be transmitted to the user terminal 140, which includes basic information about the user, through the network interface 114. The user terminal 140 may manage a sensing schedule based on the basic information and the received sensing information. The information about the schedule may be delivered to the sensor controller 110 of the sensor control system 100, and the sensor controller 110 may receive sensing information by requesting the information from the modules that includes the sensors, as described above with reference to FIG. 2, and may deliver the received sensing information to the user terminal 140.

The information input by the user and the sensing information may be combined in the user terminal 140. For example, the user terminal 140 may represent the health condition of the user using a chart, or in 2D or 3D, by combining the basic information input by the user with the received sensing information. For example, various items may be provided to the user, such as activity hours, pace counts, a work rate, an amount of burned calories, a basal metabolic rate, a weight (the amount of excess weight), body fat, body water, a pulse, information about a skeleton, muscle mass, visceral fat, energy consumption, a gait correction method, body temperature, an ovulation day in the case of a female, stress, and the like.

The user's biometric information may be managed using a program installed in the user terminal 140, and if the value indicated by the biometric information falls below a reference value, the user terminal 140 may signal the abnormality of the biometrics by outputting a warning or voice message.

For example, the user terminal 140 may provide the user with an overweight warning message and a necessary amount of exercise using the weight measured by a pressure sensor, or may provide the user with a method for correcting a gait by analyzing a Z-axis, measured by an acceleration sensor. Also, the user terminal 140 may provide a user with a warning message for rehydration and information about the amount of fluid to take by measuring body water through bioimpedance analysis, or may provide the user with information about the current body condition of the user using a heart rate sensor. In another example, the user terminal 140 may provide the user with information about when to replace shoes, a work rate and an amount of calories by measuring impact force applied to the body using a vibration sensor, information about a change in body temperature and a female's ovulation day through a temperature sensor. Also, the user terminal 140 may represent a psychological stress status and provide a warning message through a GRL sensor, and may provide information about the body condition using an insole sensor. The measurements measured by the sensors may be provided to the user terminal 140 through the sensor control system 100, as described above.

Also, the user terminal 140 may select the medical institution to which the biometric information of the user is to be transmitted. The medical institution may be automatically selected by the user terminal 140, or a user may input the selection. The user terminal 140 may periodically or occasionally (in the event of an emergency) transmit the biometric information to the server of the corresponding medical institution. The medical institution may provide information such as visiting care using the biometric information provided by the user. Particularly, in an emergency, the user's biometric information and location information may be provided not only to the medical institution but also to emergency facilities (the national emergency management agency or the like).

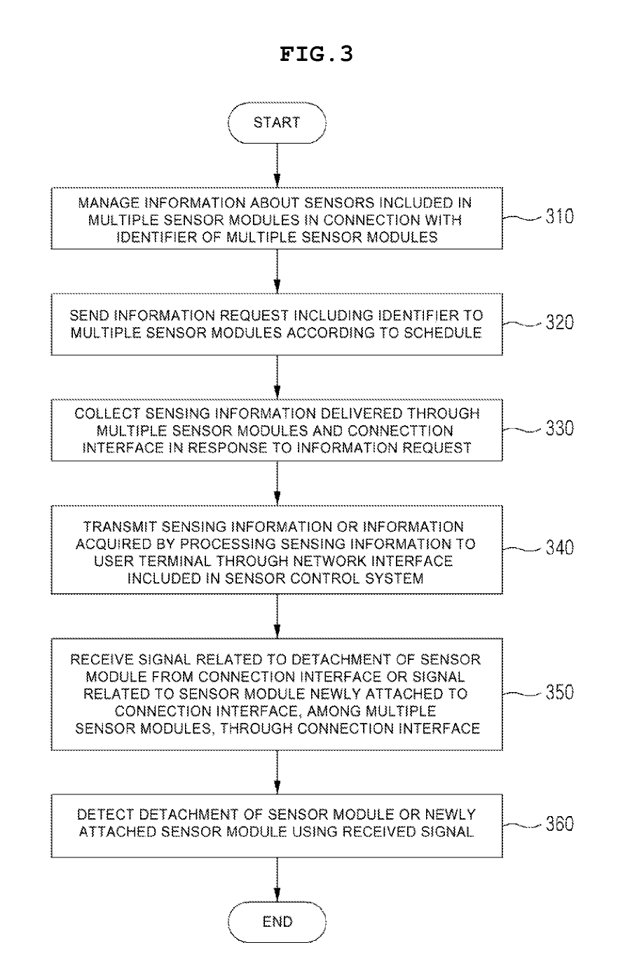

FIG. 3 is a flowchart illustrating a sensor control method in an embodiment of the present invention.

The sensor control method according to the present embodiment may be performed by the sensor control system 100.

At step 310, the sensor control system 100 may manage information about the sensors included in the multiple sensor modules in connection with the identifiers of the multiple sensor modules. The multiple sensor modules are modularized such that each of the sensor modules includes at least one sensor, and may be attached to or detached from the connection interface included in the sensor control system. Here, the module may be formed by grouping sensors according to various conditions, such as the kinds and characteristics of the sensors. The information about the sensor and the identifier thereof may be managed by the sensor controller included in the sensor control system 100.

At step 320, according to a schedule, the sensor control system 100 may send an information request that includes an identifier to the multiple sensor modules. Here, the multiple sensor modules may determine whether to reply to the information request by comparing their identifier with the identifier included in the information request. Such an information request may be sent to the multiple sensor modules by the sensor controller through the connection interface.

For example, the multiple sensor modules may include multiple digital sensor modules that are connected in series to the connection interface. Each of the multiple digital sensor modules compares its identifier with the identifier included in the information request and sends the sensing information to the sensor controller when the two identifiers are the same as each other. If the two identifiers differ from each other, the information request may be delivered to the next digital sensor module connected in series. The next digital sensor module also compares its identifier with the identifier included in the information request, and may either send the sensing information or deliver the information request to the following one. The digital sensor module may include an integrated interface for the sensors included therein. Through the integrated interface, the digital sensor module may communicate with the connection interface, or may communicate with the integrated interface of another digital sensor module connected thereto in series.

Also, the multiple sensor modules may further include an analog sensor module. The digital sensor module is attached to the connection interface in series, but each analog sensor module may be individually connected to the connection interface.

At step 330, the sensor control system 100 may collect sensing information, delivered via the multiple sensor modules and connection interface, in response to the information request. As described above, among the multiple sensor modules, the sensor module specified by the identifier included in the information request may send the sensing information in response to the information request, and the sent sensing information may be delivered to the sensor controller through the connection interface.

At step 340, the sensor control system 100 may send sensing information or information acquired by processing the sensing information to the user terminal through the network interface included in the sensor control system. Here, the schedule mentioned at step 320 may be preset using information about the kinds of sensors of the multiple sensor modules connected to the connection interface and information about the user, which is input to the user terminal.

The sensor control system 100 may be included in an object worn on a specific part of a body. If a sensor includes an electrode that requires electric contact with a user's skin, at least one of an object and a supplementary object, worn together with the object, may include conductive material between the electrode and the user's skin.

At step 350, through the connection interface, the sensor control system 100 may receive a signal related to detachment of a sensor module, among multiple sensor modules, from the connection interface, or a signal related to a sensor module that is newly attached to the connection interface, among the multiple sensor modules.

At step 360, the sensor control system 100 may recognize the detached sensor module or the newly attached sensor module using the received signal.

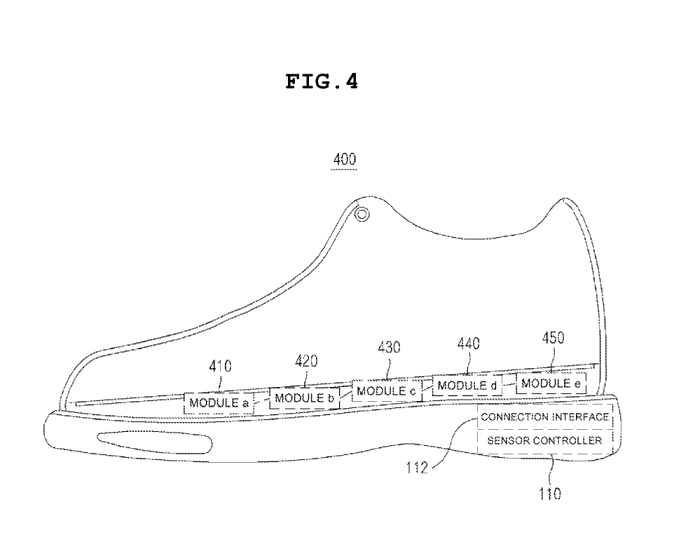

FIG. 4 is a view illustrating an example of shoes to which a sensor control system is attached in an embodiment of the present invention. FIG. 4 shows a shoe 400 that includes a sensor controller 110, a connection interface 112, and multiple sensor modules, which are module a 410, module b 420, module c 430, module d 440 and module e 450. Here, the module a 410, the module b 420, the module c 430, the module d 440 and the module e 450 are digital sensor modules, and are connected in series to the connection interface 112. If the module a 410 includes an electrode that requires electric contact with the user's skin, at least some parts (between the electrode and the user's skin) of the insole or the socks, worn by the user as a supplementary object, may be implemented using conductive material. If the user removes the module a 410, the connection interface 112 may send a signal related to the removal of the module a 410 to the sensor controller 110. Here, the sensor controller 110 may recognize that the module a 410 is detached through the received signal. If a new digital sensor module (not illustrated) is placed in the location from which the module a 410 is detached by being connected to the module b 420, the connection interface 112 enables the sensor controller 110 to detect that the new digital sensor module is attached by sending the sensor controller 110 a signal related to attachment of the new digital sensor module.

FIG. 4 merely illustrates an embodiment in which the sensor control system is included in an object. The location in which the components included in the sensor controller are arranged is not limited to this, and the object is not limited to shoes. Also, a component included in the sensor control system, such as a network interface 114, may be further included.

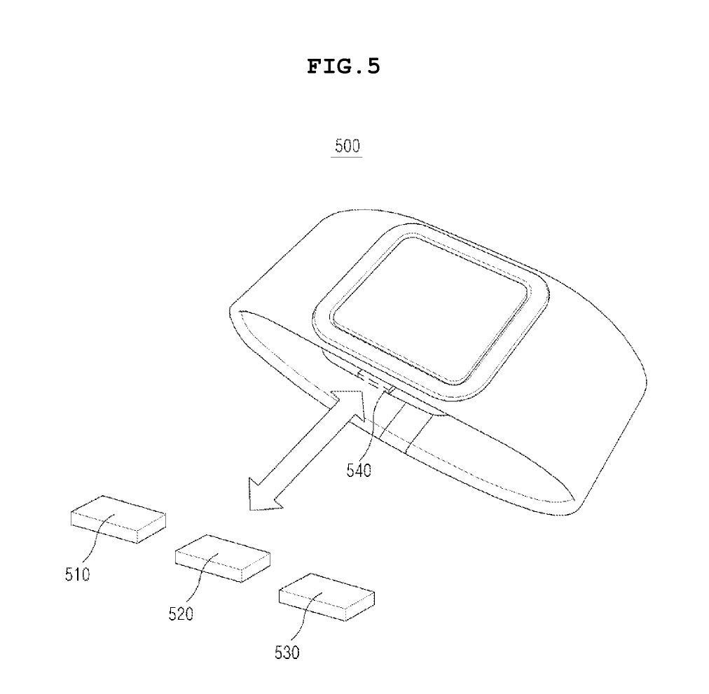

FIG. 5 is a view illustrating an example of a smart watch to which a sensor control system is attached in an embodiment of the present invention. Although not illustrated in the drawing, the smart watch 500 may include the above-mentioned sensor controller 110 and connection interface 112. Here, a user may insert a desired module 520, selected from among modules 510, 520 and 530, into a module groove 540 included in the smart watch 500. The module 520 inserted into the module groove 540 may be electrically connected to the connection interface 112, and the connection interface 112 may enable the sensor controller 110 to recognize the attached module 520 by sending a signal related to the attachment of the module 520 to the sensor controller 110.

For example, it is assumed that the module 510 is configured to include sensors for measuring bioimpedance, that the module 520 is configured to include sensors for sensing the environment, and that the module 530 is configured to include sensors for basic health care. Here, if a user wants to sense the environment, the user only needs to attach the desired module 520, selected from among the modules 510, 520 and 530, to the smart watch 500. Then, the sensor controller 110 may automatically detect the attached module 520, collect necessary information from the corresponding module 520, and process the information. Therefore, excluding selection of a module from among the modules 510, 520 and 530 and the physical connection of the modules 510, 520 and 530, the user need not care about the detection of the modules 510, 520 and 530 or information processing.

As described above, according to the embodiments of the present invention, multiple sensors are grouped, the sensors are modularized based on a group, and a module of a necessary sensor group is provided in the form of being attachable to or detachable from a wearable device, whereby a spatial limitation may be overcome. Also, a connection interface may be provided in order to enable users to easily add the sensor modules to the wearable device that includes a sensor controller, without prior knowledge. Also, an information request cycle is automatically controlled depending on the kind of sensor module attached to the sensor controller, whereby power consumption for operating the sensors may be reduced.

The system described herein may be implemented using hardware components, software components, or a combination thereof. For example, the device and components described in the embodiments may be implemented using one or more general-purpose or special-purpose computers, for example, a processor, a controller, an arithmetic logic unit (ALU), a digital signal processor, a microcomputer, a field programmable gate array (FPGA), a programmable logic unit (PLU), a microprocessor or any other device capable of responding to and executing instructions. The processing device may run an operating system (OS) and one or more software applications that run on the OS. The processing device may also access, store, manipulate, process, and create data in response to execution of the software. For convenience of understanding, the use of a single processing device is described, but those skilled in the art will understand that a processing device may comprise multiple processing elements and multiple types of processing elements. For example, a processing device may include multiple processors or a single processor and a single controller. Also, different processing configurations, such as parallel processors, are possible.

The software may include a computer program, code, instructions, or some combination thereof, and it is possible to configure processing devices or to independently or collectively instruct the processing devices to operate as desired. Software and/or data may be embodied permanently or temporarily in any type of a machine, a component, physical or virtual equipment, a computer storage medium, a device, or in a propagated signal wave in order to provide instructions or data to the processing devices or to be interpreted by the processing devices. The software may also be distributed in computer systems over a network such that the software is stored and executed in a distributed method. In particular, the software and data may be stored in one or more computer-readable recording media.

The above-described embodiments may be implemented as a program that can be executed by various computer means. In this case, the program may be recorded on a computer-readable storage medium. The computer-readable storage medium may include program instructions, data files, and data structures, either solely or in combination. Program instructions recorded on the storage medium may have been specially designed and configured for the present invention, or may be known to or available to those who have ordinary knowledge in the field of computer software. Examples of the computer-readable storage medium include all types of hardware devices specially configured to record and execute program instructions, such as magnetic media, such as a hard disk, a floppy disk, and magnetic tape, optical media, such as CD-ROM and a DVD, magneto-optical media, such as a floptical disk, ROM, RAM, and flash memory. Examples of the program instructions include machine code, such as code created by a compiler, and high-level language code executable by a computer using an interpreter. The hardware devices may be configured to operate as one or more software modules in order to perform the operation of the present invention, and vice versa.

Although the preferred embodiments of the present invention have been disclosed for illustrative purposes, those skilled in the art will appreciate that various modifications, additions and substitutions are possible, without departing from the scope and spirit of the invention as disclosed in the accompanying claims. For example, even if the described techniques are performed in a different order, the described components, such as systems, architectures, devices, and circuits, are combined or coupled with other components by a method different from the described methods, or the described components are replaced with other components or equivalents, the results are still to be understood as falling within the scope of the present invention.

Therefore, other implementations, embodiments, and modifications equivalent to the claims are included in the scope of the claims set forth below.

1. A sensor control system, comprising:
multiple sensor modules, each of the multiple sensor modules being modularized to include at least one sensor, being attached to or detached from a connection interface, and sending information sensed by the at least one sensor to a sensor controller through the connection interface;
the connection interface, which the multiple sensor modules are attached to or detached from, and which relays communication between the sensor controller and the multiple sensor modules; and
the sensor controller for requesting information from the multiple sensor modules through the connection interface according to a schedule and for processing sensing information received from the multiple sensor modules,
wherein the sensor control system is included in an object worn on a specific part of a human body.

2. The sensor control system of claim 1, wherein the multiple sensor modules comprise:
one or more analog sensor modules individually attached to or detached from the connection interface; and
multiple digital sensor modules attached to or detached from the connection interface in series,
wherein each of the digital sensor modules comprises an integrated interface for a sensor included therein, and communicates with the connection interface through the integrated interface, or communicates with an integrated interface of another digital sensor module attached thereto in series through the integrated interface.

3. The sensor control system of claim 1, wherein the sensor controller is configured to:
manage information about the at least one sensor included in each of the multiple sensor modules in connection with an identifier of the sensor module; and
send an information request that includes the identifier to the multiple sensor modules through the connection interface.

4. The sensor control system of claim 1, wherein:
the multiple sensor modules comprise multiple digital sensor modules attached to the connection interface in series, and
in response to an information request sent by the sensor controller, each of the multiple digital sensor modules compares its identifier with an identifier included in the information request and sends sensing information to the sensor controller if the two identifiers are identical to each other, or delivers the information request to a next digital sensor module attached thereto in series if the two identifiers differ from each other.

5. The sensor control system of claim 1, wherein:
the connection interface sends the sensor controller a signal related to a sensor module detached from the connection interface or a signal related to a sensor module newly attached to the connection interface, among the multiple sensor modules; and
the sensor controller detects the detached sensor module or the newly attached sensor module using the received signal.

6. The sensor control system of claim 1, wherein
if the at least one sensor includes an electrode that requires electric contact with a skin of a user, at least one of the object and a supplementary object, worn together with the object, includes conductive material between the electrode and the skin of the user.

7. The sensor control system of claim 1, further comprising a network interface for transmitting the sensing information or information acquired by processing the sensing information to a user terminal,
wherein the schedule is preset using information about a kind of a sensor of the multiple sensor modules attached to the connection interface and information about a user, which is input to the user terminal.

8. A sensor control method of a sensor control system, comprising:
managing information about sensors included in multiple sensor modules in connection with identifiers of the multiple sensor modules;
sending an information request that includes the identifier to the multiple sensor modules according to a schedule;
determining, by the multiple sensor modules, whether to reply to the information request by comparing their identifier with the identifier included in the information request; and
collecting sensing information delivered through the multiple sensor modules and a connection interface in response to the information request,
wherein the sensor control system is included in an object worn on a specific part of a human body.

9. The sensor control method of claim 8, further comprising,
sending the sensing information or information acquired by processing the sensing information to the user terminal through a network interface included in the sensor control system,
wherein the schedule is preset using information about a kind of a sensor of the multiple sensor modules attached to the connection interface and information about a user, which is input to the user terminal.

10. The sensor control method of claim 8, wherein:
the multiple sensor modules comprise multiple digital sensor modules connected in series to the connection interface; and
in response to the information request sent by the sensor controller, each of the multiple digital sensor modules compares its identifier with the identifier included in the information request and sends sensing information to the sensor controller if the two identifiers are identical to each other, or delivers the information request to a next digital sensor module attached thereto in series if the two identifiers differ from each other.

11. The sensor control method of claim 9, further comprising:
receiving, through the connection interface, a signal related to a sensor module detached from the connection interface or a signal related to a sensor module newly attached to the connection interface, among the multiple sensor modules; and
detecting the detached sensor module or the newly attached sensor module using the received signal.
